(19) United States
(12) Patent Application Publication
(10) Pub. No.: US 2008/0300537 A1
(43) Pub. Date: Dec. 4, 2008

(54) METHOD AND SYSTEM FOR STEERING A CATHETER END IN MULTIPLE PLANES

(76) Inventor: David Allen Bowman, Marietta, GA (US)

Correspondence Address: John Doughty, 42 Creekside Court, Acworth, GA, 30101, US

(21) Appl. No.: 12/132,102

(22) Filed: Jun. 3, 2008

Related U.S. Application Data
(60) Provisional application No. 60/941,690, filed on Jun. 3, 2007.

(51) Int. Cl. A61M 25/092

(52) U.S. Cl. 604/95.04

(57) ABSTRACT

A catheter extrusion includes one or more extra steering wire lumens to route one or more steering wires that facilitate steering of a distal end in more than one plane. To make room in an extrusion, for extra steering wires, the extrusion may define a smaller operational lumen. Alternatively, a designer/manufacturer may increase the size of the extrusion from to facilitate one or more extra operational lumen(s).

An existing Naviscope pistol grip assembly is modified to include a second plane steering wheel than transfers a pressing force on one or more buttons thereof into rotational motion that causes corresponding tension in respective one or more a steering wire(s) connected thereto. The additional steering wires are guided by existing steering guide wheels used for two-wire steering.

### CROSS REFERENCE TO RELATED APPLICATION

This application claims priority under 35 U.S.C. 119(e) to U.S. provisional patent application No. 60/941,690 entitled “System for multi-directional steering,” which was filed Jun. 3, 2007, and is incorporated herein by reference in its entirety

### BACKGROUND

Catheters used in the medical industry, such as catheter products produced by Myelotec, Inc. for example, steer a distal end by manipulating stainless steel wires anchored into the distal tip of a plastic extrusion. FIG. 1 illustrates a catheter assembly 2 comprising extrusion 3 and distal end 4. Distal end 4 may include a lens and light carrying means, such as optical fiber, that facilitates a user viewing tissue and organs of a human, or animal, body. Applying tension to the proximal end 5 of either wire 6 causes distal end 4 of extrusion 3 to deflect in the direction of the wire 6, to which tension was applied, relative to the centerline of extrusion 3. For example, FIG. 2 illustrates that tension has been applied to rightmost steering wire 6R causing distal end 4 to deflect to the right. Similarly, applying tension to steering wire 6L (the figure does not illustrate this scenario for clarity) would cause distal end 4 of extrusion 3 to deflect to the left, or the opposite direction as that shown in the figure, but in the same plane as the movement depicted in FIG. 2.

In a two-wire configuration, such as the Naviscope® product line sold by Myelotec, Inc., a user may steer in only one plane at a time. Although the steering plane may be rotated 90° via a steering yoke and external, collar from horizontal to vertical (See FIG. 3), compound movements, such as left-to-right sweeps while pointing downward, cannot be performed, using two steering wires. As shown in FIG. 3, yoke bearing 8 receives rotation yoke 10 and rotation pin 11 protrudes through rotation slot 13. When housing halves 15 and 17 are mated together, collar 22 slides over the end of the mated housing halves and engages pin 11. Thus, a user gripping collar 22 can rotate extrusion 3 by turning collar 22. The extent of slot 13 limits the amount of rotation so that wires 6R and 6L remain untangled. When steering bearing 16 has received axle 18 of steering wheel 20, manipulating buttons 12 and 14 causes the distal end of extrusion 3 to deflect correspondingly. For example, pressing button 14 20 induces tension in steering wire 6R, which causes the distal end to deflect to the right as described above in connection with the discussion of FIG. 2. Similarly, pressing button 12 would induce tension in steering wire 6L and cause the distal, end to deflect to the left.

A user may be able to rotate the steering plane (via collar 22) and re-point distal end 4 toward a desired location within a body, but only if the desired location falls within the area depicted in FIG. 7A. Should the location fall outside the possible steering planes (the point depicted with an X in FIG. 7A e.g.) the user must rotate housing halves 15 and 17 for viewing. However, since the optical fiber is attached to the housing, the image seen will be tilted with respect to the previous orientation of the image. Thus, there is a need for a catheter steering device that can steer a distal end of a catheter extrusion in multiple planes rather than one plane without causing image tilt as occurs when the distal end is rotated via the housing halves rather than being steered,

### DESCRIPTION OF THE DRAWINGS

FIG. 1 illustrates a catheter extrusion for facilitating two-way steering of a distal end.

FIG. 2 illustrates the distal end of a catheter extrusion deflected in a rightward direction.

FIG. 3 illustrates a Naviscope pistol grip assembly for facilitating two-way steering of a catheter extrusion distal end.

FIG. 4A illustrates a sectional view of a catheter extrusion for facilitating two-way steering.

FIG. 4B illustrates a sectional view of a catheter extrusion for facilitating three-way steering.

FIG. 5 illustrates a catheter extrusion for facilitating three-way steering of a distal end.

FIG. 6 illustrates a Naviscope pistol grip assembly modified to facilitate three-way steering of a catheter extrusion distal end.

FIG. 7A illustrates the possible steering planes of a two-wire steered extrusion.

FIG. 7B illustrates possible planes of a three-way steered extrusion.

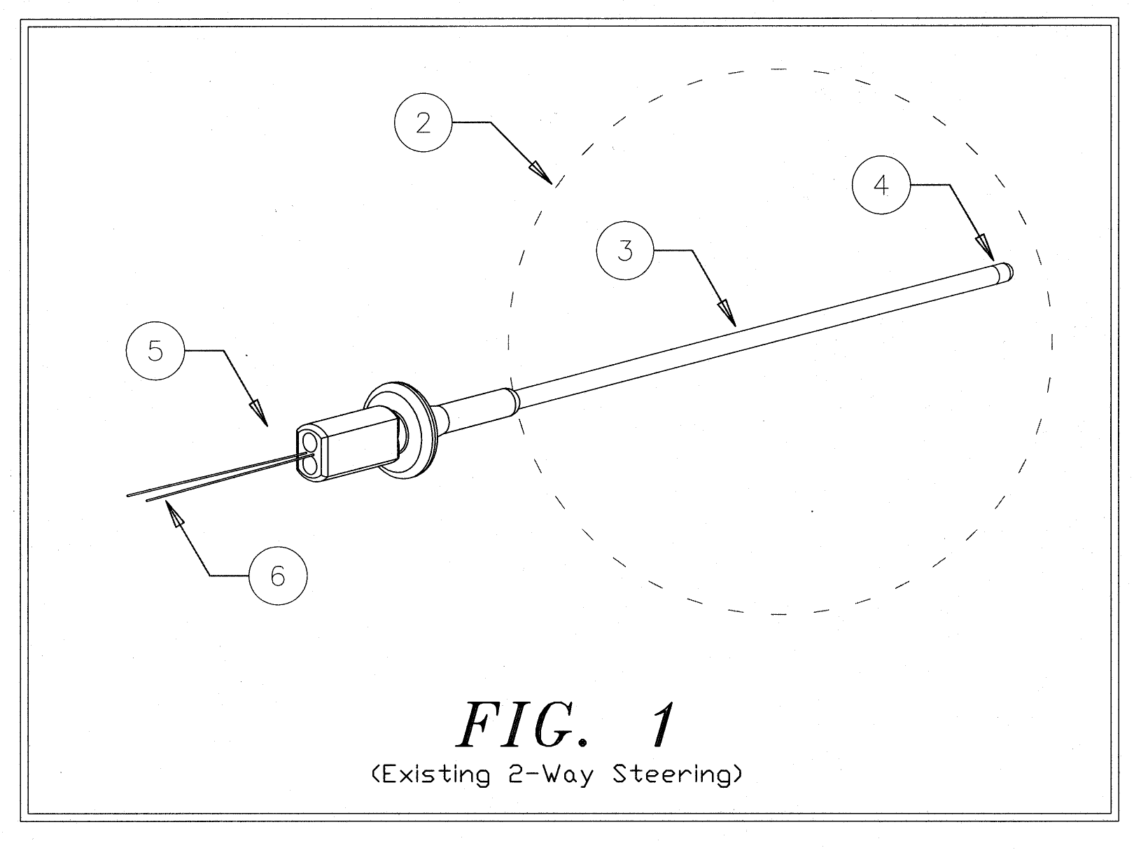

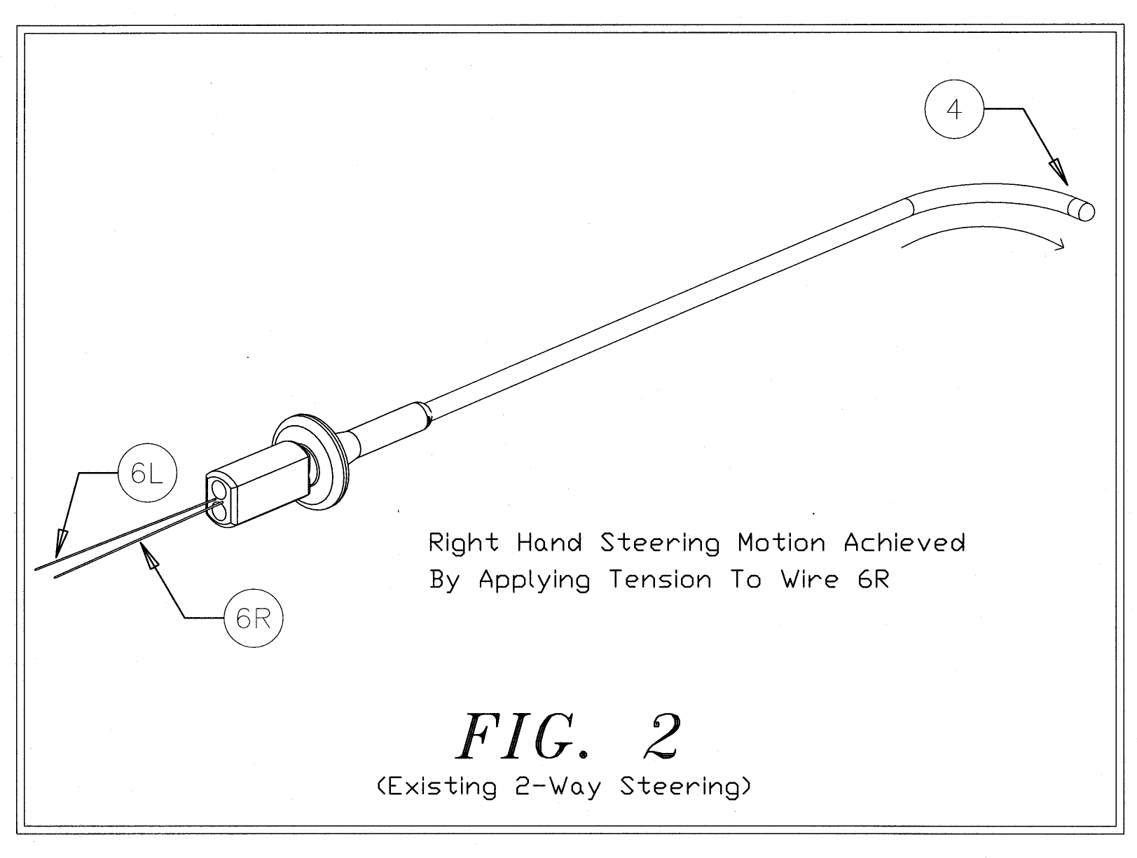

### DETAILED DESCRIPTION

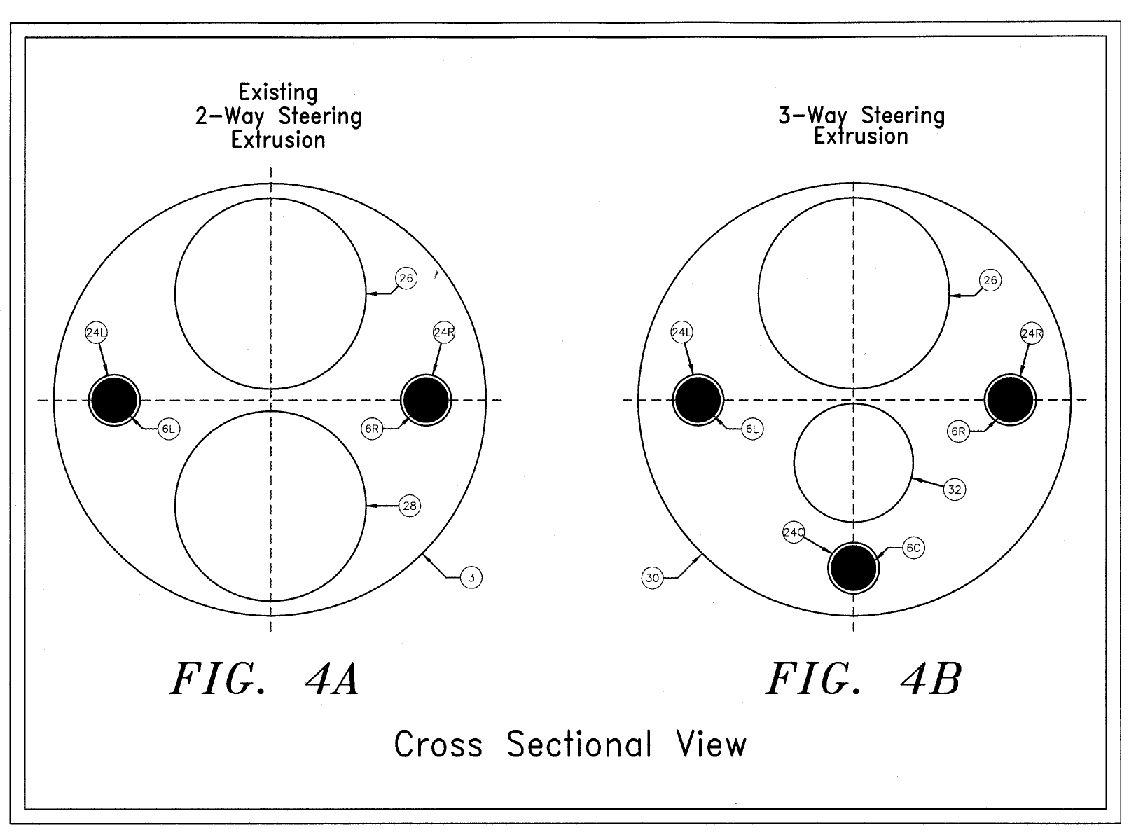

Turning now to the figures, FIG. 4 illustrates an aspect by comparing a sectional plan view of a catheter extrusion 3 for two way steering that uses two steering wires in FIG. 4A with a sectional plan view in FIG. 4B of a catheter that facilitates multiway steering using three steering wires. As discussed in connection with previous figures, steering wires 6R and 6L enter extrusion 3 through respective steering wire lumens 24R and 24L. Operational lumens 26 and 28 are shown larger than the steering wire lumens 24R and 24 L because they facilitate operational items such as, for example, fiber for passing light to and from a distal end of extrusion 3 and possibly a tube carrying air or wires for manipulating a cutter for abscising tissue. In the two steering wire system facilitated by the section of extrusion 3 shown in FIG. 4A, lumens 26 and 28, although of equal diameter, are sized so that they equitably share the sectional area of the section shown in the figure with lumens 24L and 24R.

Turing now to FIG. 4B, extrusion 30 defines center steering wire lumen 24C. Lumen 24C receives and routes steering wire 6C, which transfers force from a hand grip to a distal end of extrusion 30, causing the distal end to move in a plane orthogonal to the plane within which wires 6L and 6R cause movement. Since only center wire 6C causes motion in what will be referred to herein as a second plane (the first plane being the plane in which tension in wires 61, and 6R cause motion), the motion in the second plane will occur in only one direction away from a relaxed, or straight-extending, position of the distal end of extrusion 30. To accommodate the extra area of the illustrated section of extrusion 30, the extrusion defines smaller lumen 32 rather than lumen 28, which is similarly sized as lumen 26. Extrusion 30 defines the size of smaller lumen 32 to make room for the addition of steering wire lumen 24C while still being large enough in diameter to accommodate items described above, such as light fiber or possibly wires for manipulating a biopsy tissue sample collector. Alternatively, a designer/manufacturer may increase the size of the extrusion from, for example, 3.0 mm to 3.3 mm, to facilitate the extra operational lumen(s) of suitable size.

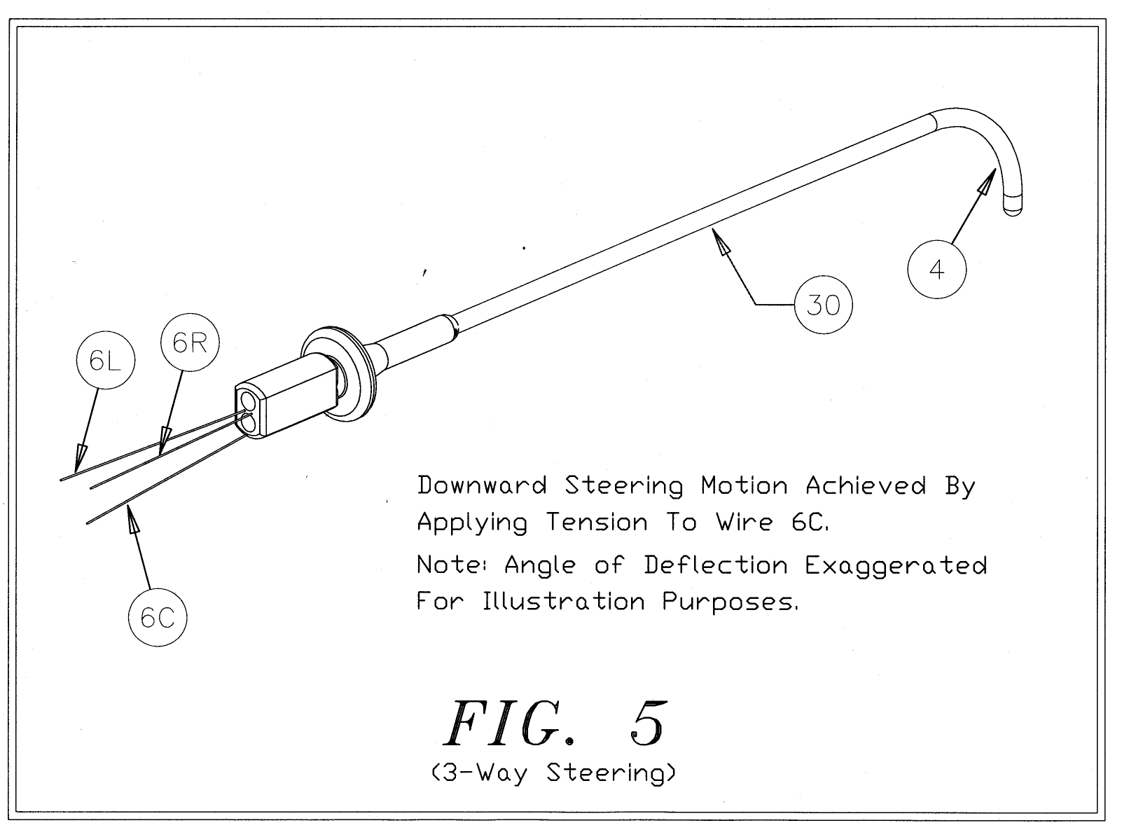

Turning now to FIG. 5, the figure illustrates steering wires 6L, 6R and 6C being routed through corresponding steering wire lumens defined by extrusion 30. Tension in center steering wire 6C causes distal tip to deflect downward as shown in the figure. It will be appreciated that a fourth steering wire and corresponding steering wire lumen could be added to cause motion of distal end 4 in the up direction as well as the down direction.

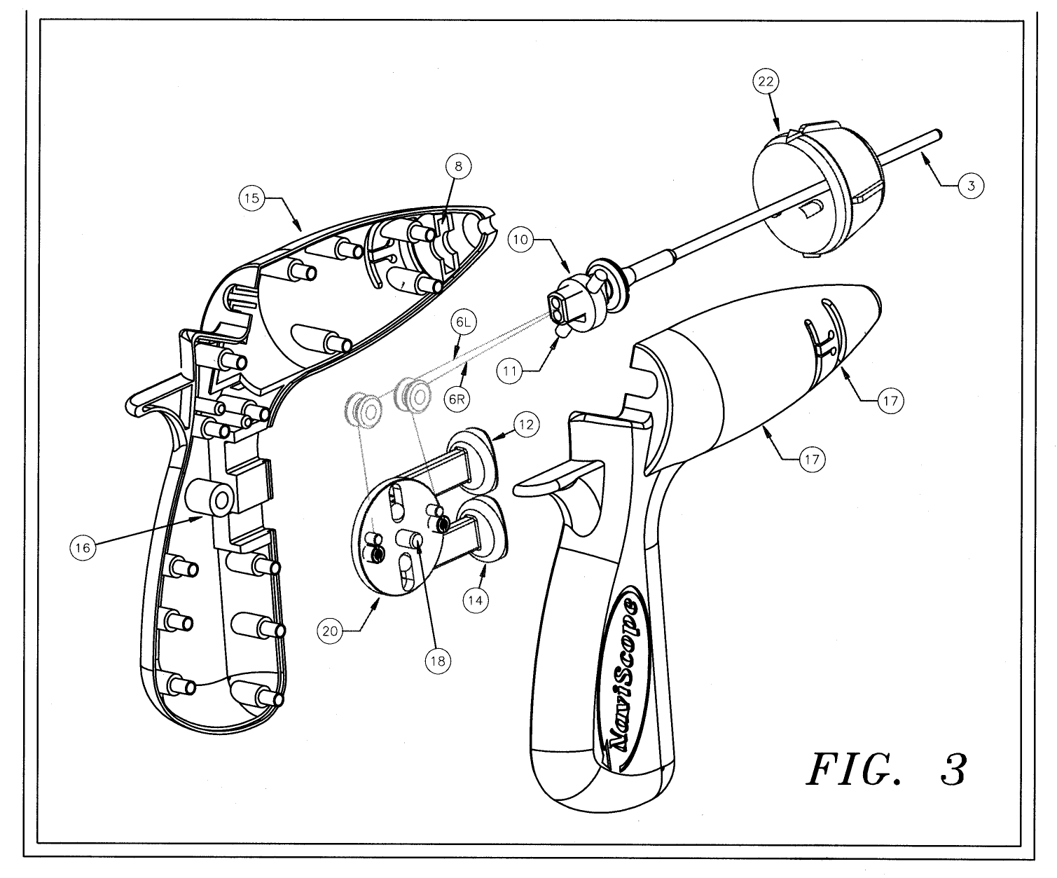

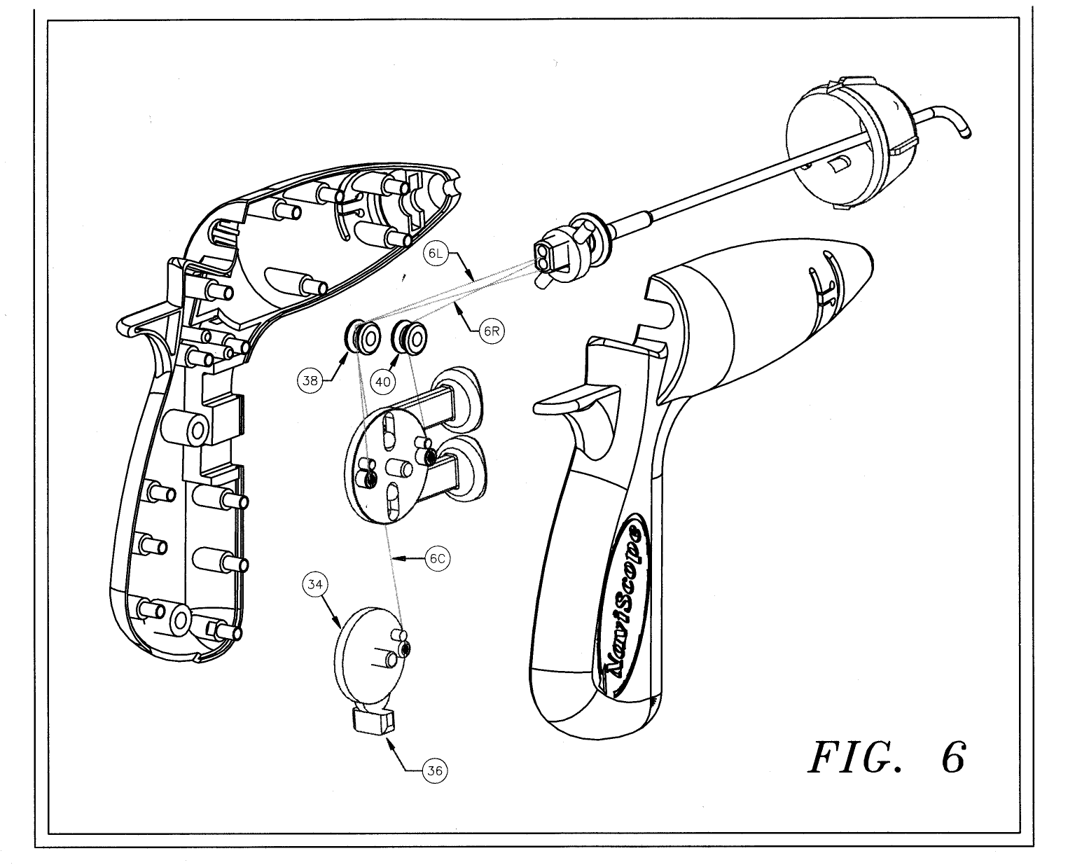

Turning now to FIG. 6, the figure illustrates a second plane steering dial 34 added to the Naviscope assembly that is shown in FIG. 3. Manipulation of steering tab 36 in the clockwise direction causes tension in center steering wire 6C, which causes motion in the down direction of distal tip 4. Center steering wire 6C shares guide wheel 38 with left steering wire 6L, which is already present in a two wire steering system, thus reducing the number of addition parts to facilitate three wire steering as compared to two wires steering. Steering wire 6R continues to use guide wheel 40 as it does in a two wire steering arrangement.

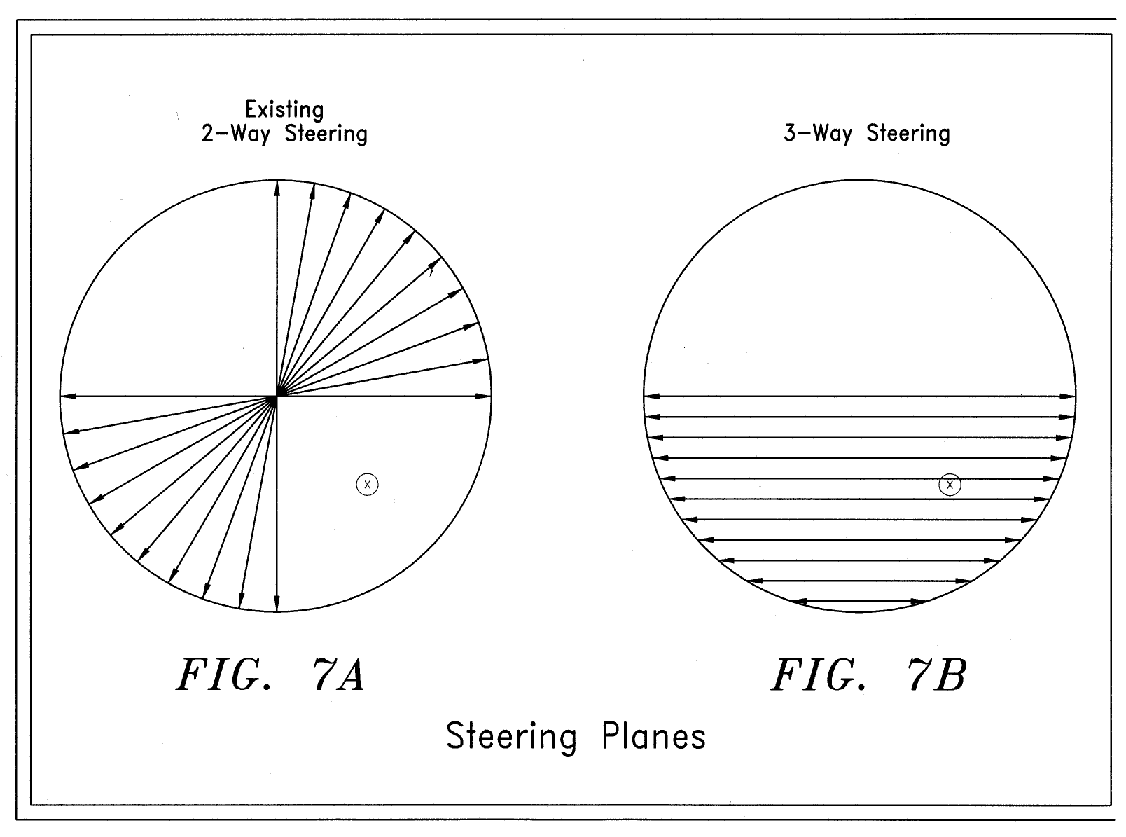

Turning now to FIG. 7B, for the figure shows that the point depicted with an X can be viewed in proper orientation by first deflecting distal end 4 downward, and then steering left to right as normal. Thus, image orientation does not change as a user steers extrusion 3 as compared to the image orientation change that occurs when a user rotates the steering handgrip housing in a two-wire steering system.

What is claimed is:

1. A device for steering a catheter scope distal end, comprising:
means for steering the distal end in a first plane; and
means for steering the distal end in a second plane, the second plane being non-parallel to the first plane,

2. The device of claim 1 wherein the device locates the means for steering the probe end in the second plane orthogonal to the location, of the means for steering the probe end in the first pane such that the second plane is orthogonal to the first plane,

3. The device of claim 2 including right and left steering lumens that locate steering wires for steering in the first plane and a steering lumen that locates a center steering wires for steering in the second plane, wherein the rights and left steering lumens are located in a catheter extrusion, substantially 180 degrees from one another with respect to the circumference of the extrusion, and the center lumen is located substantially ninety degrees from either the right or the left steering lumen.

4. The device of claim 3 wherein the right, the left and the center steering lumens are disposed substantially equidistant from a centerline of the catheter extrusion.

5. The device of claim 1 wherein the means for steering include steering wires.

6. The device of claim 1 including operational lumens each of the same diameter.

7. The device of claim 1 including operational lumens wherein each one of the operational lumens is of a different diameter than the diameter of another one of the operational lumens.

8. A method for using a steering device to steer a catheter scope distal end, comprising:
manipulating buttons located by a handgrip of the steering device to cause means for steering the distal end in a first plane to steer the distal end in the first plane; and
manipulating a steering tab located by the handgrip to cause means for steering the distal end in a second plane to steer the distal end in the second plane.

9. The method of claim 8 wherein the steering device locates the means for steering the probe end in the second plane orthogonal to the location of the means for steering the probe end in the first pane such that the second plane is orthogonal to the first plane.

10. The method of claim 9 wherein the steering device includes right and left steering lumens that locate steering wires for steering in the first plane and a steering lumen that locates a center steering wires for steering in the second plane, wherein the rights and left steering lumens are located in a catheter extrusion substantially 180 degrees from one another with respect to the circumference of the extrusion, and the center lumen is located substantially ninety degrees from either the right or the left steering lumen.

11. The method of claim 10 wherein operation of the steering device causes the steering wires to exert force through the steering lumens wherein right, the left and the center steering lumens are disposed substantially equidistant from a centerline of the catheter extrusion.

12. The method of claim 8 wherein the means for steering includes steering wires.

13. The method of claim 8 wherein the steering device is used to transmit force from an operator to the distal end via steering means that are routed through operational lumens that are each of the same diameter.

14. The method of claim 8 wherein the steering device is used to transmit force from an operator to the distal end via steering means that are routed through operational lumens wherein at least one of the operational lumens is of a different diameter than the diameter of another one of the operational lumens.

15. A catheter extrusion configured for coupling to a steering device for steering a distal end of the extrusion in more than one plane, the extrusion defining steering lumens wherein a right, a left and a center steering lumen are disposed substantially equidistant from a centerline of the catheter extrusion.

16. The extrusion of claim 15 further comprising operational lumens, wherein the operational lumens are each of the same diameter.

17. The extrusion of claim 15 further comprising operational lumens, wherein at least one of the operational lumens is of a different diameter that at least one of the other operational lumens.
